(19) United States
(12) Patent Application Publication
(10) Pub. No.: US 2018/0295296 A1
(43) Pub. Date: Oct. 11, 2018

(54) PORTABLE MULTISPECTRAL IMAGING DEVICE AND METHOD OF REDUCING INTERFERENCE OF IMAGES THEREOF

(71) Applicant: EXPANTRUM OPTOELECTRONICS, SHANGHAI (CN)

(72) Inventor: Zhongshou HUANG, SHANGHAI (CN)

(73) Assignee: EXPANTRUM OPTOELECTRONICS, SHANGHAI (CN)

(21) Appl. No.: 15/894,803

(22) Filed: Feb. 12, 2018

(30) Foreign Application Priority Data
Apr. 5, 2017 (CN) 201710233037.2

(51) Int. Cl. H04N 5/33; G02F 1/1368; H04N 5/225; H04N 5/374; H04N 5/372; A61B 5/00

(52) U.S. Cl. CPC H04N 5/332; G02F 1/1368; H04N 5/2256; H04N 5/374; G02F 2001/136222; A61B 5/0075; A61B 5/0077; A61B 5/489; H04N 5/372

(57) ABSTRACT

A portable multispectral imaging and projecting device includes a liquid crystal light valve configured to modulate, in response to an image generated by a processor, intensity of light passing through the liquid crystal light valve. The light is emitted by a light source module and the liquid crystal valve projects the modulated light and the image to a target area. The light source module includes a plurality of visible light sources and at least one infrared light source. The image is generated in response to an infrared light image and a visible light image, or the infrared light image, wherein the images are acquired by a photoelectric sensor of an image acquiring module.

### CROSS REFERENCE

This application is based upon and claims the benefit of priority of Chinese Patent Applications No. 201710233037.2 filed on Apr. 5, 2017, the entire contents thereof are incorporated herein by reference.

### TECHNICAL FIELD

The present disclosure relates to the field of locating blood vessels, specifically to a portable multispectral imaging device and a method of reducing interferences of blood vessels images.

### BACKGROUND

Subcutaneous structures and blood vessels under skin are barely visible for naked eyes. In order to identify and locate the subcutaneous structures and blood vessels, medical doctors have to rely on the external outline of human body and their anatomic knowledge.

The blood vessels, including veins and arteries, are below the epidermis, and even covered by subcutaneous fat. Visible light imaging signals, reflected back from subcutaneous structures and blood vessels under the visible light illumination, are extremely faint and mixed with scattered light and various phantoms. Before puncturing, in order to make the blood vessels more visible, medical doctors often ask patients to clench their fists or flap the skin above the blood vessel. However, the visibility of subcutaneous blood vessels is still not satisfied in most cases accompany with ages, or thickness of subcutaneous fat of patients. Injection relying on the vague images of blood vessels often results misalignment of the puncture, causing pain in patients, delaying optimal time for medical treatments, and even triggering injection incident.

In addition to drawing blood and injections in various occasions, blood vessels are also needed to be accurately located during acupuncture and medical surgeries.

### SUMMARY

The primary purpose of the present disclosure is to provide a portable multispectral imaging device and a method of acquiring images of blood vessels and subcutaneous structures thereof. Therefore, the medical doctors are able to accurately detect the positions of subcutaneous structures and blood vessels which allow the medical doctors to have sufficient information for applying diagnosis and treatments.

In one embodiment of the present disclosure, a portable multispectral imaging and projecting device comprises a liquid crystal light valve configured to modulate, in response to an image generated by a processor, intensity of light, emitted by a light source module and passing through the liquid crystal light valve, and project the modulated light and the image to a target area. The light source module includes a plurality of visible light sources and at least one infrared light source and the image is generated in response to an infrared light image and a visible light image, or the infrared image. The images are acquired by a photoelectric sensor of an image acquiring module. Moreover, the light source module is configured to project visible light and infrared light.

In one embodiment of the present disclosure, a method of multispectral imaging and projecting comprises steps of projecting, by a light source module, visible light and infrared light to a target area via a liquid crystal light valve and a lens module; acquiring, by a photoelectric sensor of an image acquiring module, a visible light image and an infrared image of the target area; generating, by a processor, an image of the target area in response to the visible light image and the infrared image; modulating, by the liquid crystal light valve, intensity of the visible light, projected by the light source, in response to the image of the target area; and projecting, by the liquid crystal light valve and the lens module, the image presented by the modulated visible light to the target area.

In one embodiment of the present disclosure, a method of multispectral imaging and projecting comprises steps of projecting, by a light source module, first infrared light to a target area via a liquid crystal light valve and a lens module, and acquiring, by a photoelectric sensor of an image acquiring module, a first infrared image of the target area, wherein the light source module includes a plurality of visible light sources and at least one infrared light source; processing, by the processor, the first infrared image and transmitting, by the processor, the processed first infrared image to the liquid crystal light valve; modulating, by the liquid crystal light valve, intensity of the infrared light, projected by the light source module, in response to the processed first infrared image; projecting, by the liquid crystal light valve and the lens module, the second infrared light to the target area, and acquiring, by the photoelectric sensor of an image acquiring module, a second infrared image of the target area; processing, by the processor, the second infrared image and transmitting, by the processor, the processed second infrared image to the liquid crystal light valve; modulating, by the liquid crystal light valve, intensity of a visible light, projected by the light source module, in response to the processed second infrared image; projecting, by the liquid crystal light valve and the lens module, the processed second infrared image presented by the modulated visible light, to the target area.

### BRIEF DESCRIPTION OF THE FIGURES

Embodiments of the present disclosure will now be described, by way of example only, with reference to the accompanying schematic drawings in which corresponding reference symbols indicate corresponding parts, and in which:

FIG. 1 shows a schematic functional block diagram of a multispectral imaging and projection device of one embodiment of the present disclosure;

FIG. 2 shows a schematic view of a light source module of one embodiment of the present disclosure;

FIG. 3 shows a schematic view of a light source module of one embodiment of the present disclosure;

FIG. 4 shows a schematic view of a light source module of one embodiment of the present disclosure;

FIG. 5 shows schematic cross-sectional view of a liquid crystal light valve of one embodiment of the present disclosure;

FIG. 6 shows a transmittance diagram of red filter, green filter and blue filter for light radiations with various wavelengths;

FIG. 7 show a time sequence of a portable multispectral imaging and projecting device of one embodiment of the present disclosure;

FIG. 8 shows a multispectral imaging and projecting method implemented on portable multispectral imaging and projecting device of one embodiment of the present disclosure;

FIG. 9 shows a time sequence of a portable multispectral imaging and projecting device of one embodiment of the present disclosure;

FIG. 10 shows a multispectral imaging and projecting method implemented on portable multispectral imaging and projecting device of one embodiment of the present disclosure; and

FIG. 11 shows infrared light intensity distribution diagrams of one embodiment of the present disclosure.

### DETAILED DESCRIPTION

In the following, embodiments of the present disclosure will be described in detail referring to figures. The concept and its realizations of the present disclosure can be implemented in a plurality of forms, and should not be understood to be limited to the embodiments described hereafter. In contrary, these embodiments are provided to make the present disclosure more comprehensive and understandable, and so the conception of the embodiments can be conveyed to the technicians in the art fully. Same reference signs in the figures refer to same or similar structures, so repeated description of them will be omitted.

The features, structures or characteristics described can be combined in any appropriate way in one or more embodiments. In the description below, many specific details are provided to explain the embodiments of the present disclosure fully. However, the technicians in the art should realize that, without one or more of the specific details, or adopting other methods, components, materials etc., the technical proposal of the present disclosure can still be realized. In certain conditions, structures, materials or operations well known are not shown or described in detail so as not to obfuscate the present disclosure.

The technical contents of the present disclosure will be further described below with reference to the figures and embodiments.

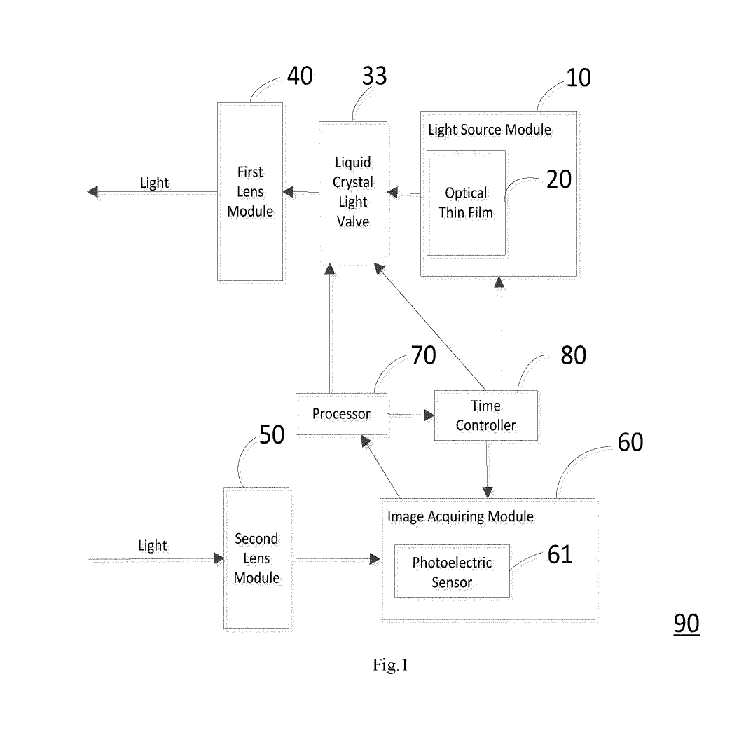

FIG. 1 shows schematic functional diagrams of a portable multispectral imaging and projection device of one embodiment of the present disclosure. As shown in FIG. 1, the portable multispectral imaging and projection device 90 includes a liquid crystal light valve 33 configured to modulate intensity of light, emitted by a light source module 10 and passing through the liquid crystal light valve 33, in response to an image generated by a processor 70. The liquid crystal light valve 33 is then project the image presented by the modulated light 15 to a target area (not shown) via a first lens module 40. In this embodiment, the first lens module 40 includes at least one lens. Moreover, in this embodiment, the light source module 10 includes a plurality of visible light sources and at least one infrared source. In some embodiments, the light source module 10 further includes an optical thin film 20 configured to form homogenous plan light.

The image is generated in response to visible light image and infrared image or infrared image, which are acquired by a photoelectric sensor 61 of the image acquiring module 60. In this embodiment, the second lens module 50 includes at least one lens configured to focus light to the photoelectric sensor 61. Moreover, the photoelectric sensor 61 is driven by a thin film transistor (TFT) array.

In this embodiment, the photoelectric sensor 61 includes a charge-coupled device (CCD) imaging sensor having a photodiode. In some embodiments, the photoelectric sensor 61 includes a CCD having a bipolar transistor. In some embodiments, the photoelectric sensor 61 includes a complementary metal oxide semiconductor (CMOS) imaging sensor having a photodiode. In some embodiments, the photoelectric sensor 61 includes a CMOS having a bipolar transistor.

Furthermore, the image acquiring module 60 includes a CCD integrated with a semiconductor photo-electric conversion film. In some embodiments, the image acquiring module 60 includes a CMOS integrated with the semiconductor photo-electric conversion film. Moreover, the semiconductor photo-electric conversion film includes amorphous silicon or a metal oxide semiconductor.

As shown in FIG. 1, in this embodiment, the multispectral imaging and projection device 90 further includes a time controller 80 configured to control, in response to a projecting control signal transmitted from the processor 70, the light source module 10 to simultaneously or alternatively emits light radiations. Furthermore, the time controller 80 is configured to control the liquid crystal light valve 33, to output projecting images generated by the processor 70. In this embodiment, the time controller 80 is configured to control the image acquiring module 60 in response to an acquiring signal transmitted from the processor 70. Moreover, the center of the light source module 10, the center of optical thin film 20, the center of the liquid crystal light valve 33, and the center of first lens module 40 are aligned on an optical axis.

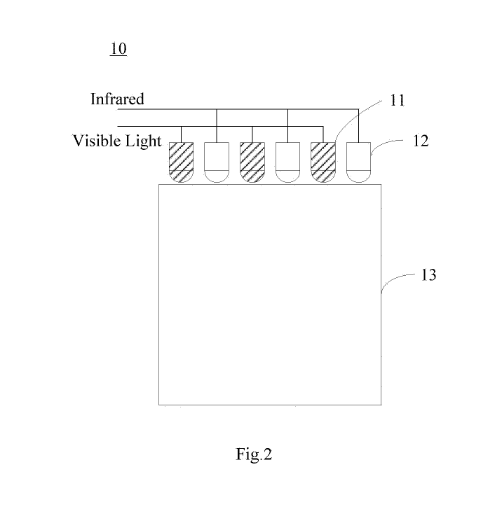

FIG. 2 shows a schematic view of a light source module 10 of one embodiment of the present disclosure. As shown in FIG. 2, in this embodiment, the light source module 10 includes a plurality of visible light sources 11, a plurality of infrared light sources 12 and a light guide panel 13, wherein a visible light source and an infrared light source are alternatively arranged alone a side of the light guide panel 13. In this embodiment, the light guide panel includes a dot structure 22 which allow light radiations of the light sources 11 and 12 to go alone a direction orthogonal to the dot structure 22.

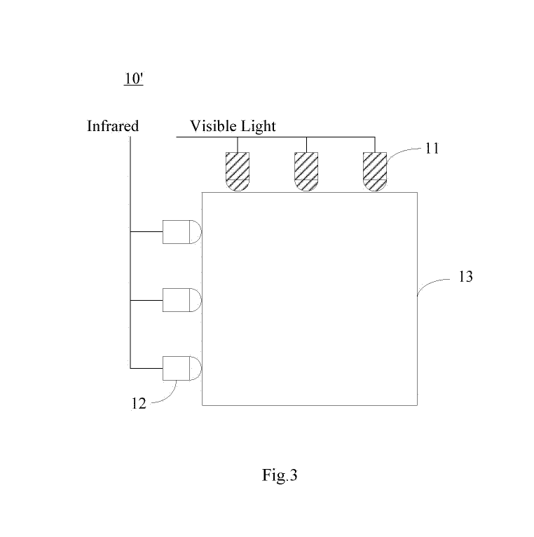

FIG. 3 shows a schematic view of a light source module 10′ of one embodiment of the present disclosure. As shown in FIG. 3, the light source module 10′ is similar to the light source module 10 of FIG. 2 but the visible light sources 11 are arranged alone a first side 33 of the light guide panel 13 and the infrared light sources 12 are arranged alone a second side 34 of the light guide panel 13, wherein, as shown in FIG. 3, the first side 33 is adjacent to the second side 34.

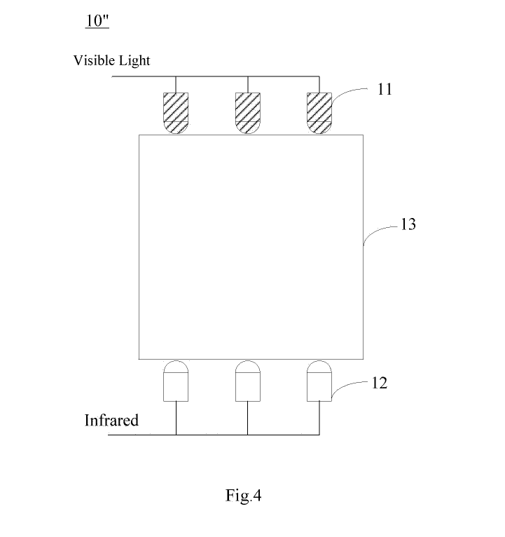

FIG. 4 shows a schematic view of a light source module 10″ of one embodiment of the present disclosure. As shown in FIG. 4, the light source module 10″ of FIG. 4 is similar to the light source module 10 of FIG. 2 but the visible light sources 11 are arranged alone a first side 43 of the light guide panel 13 and the infrared light sources 12 are arranged alone a second side 44 of the light guide panel 13, wherein, as shown in FIG. 3, the first side 43 is opposite to the second side 44.

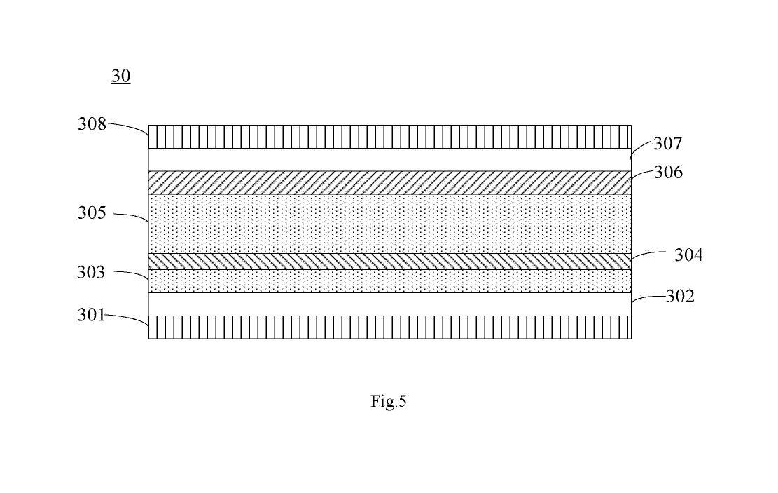

FIG. 5 shows schematic cross-sectional view of a liquid crystal light valve 33 of one embodiment of the present disclosure. In this embodiment, the liquid crystal light valve 33 includes a lower polarizing filter 301, a lower transparent glass substrate 302, a pixel array 303 driven by a thin film transistor array (not shown), a lower transparent electrode 304, a liquid crystal layer 305, an upper transparent electrode 306, an upper transparent glass substrate 307 and an upper polarizing filter 308. In this embodiment, the thickness of the liquid crystal layer 305 is approximately 3 μm to 5 μm.

In this embodiment, the intensity of light, passing through the liquid crystal light valve 33, is modulated by adjusting voltage applied on the liquid crystal layer 305 of the liquid crystal light valve 33. Moreover, in some embodiments, the liquid crystal light valve 33 is configured to modulate, e.g. two dimensional modulations, the intensity of the light in response to the image generated by the processor.

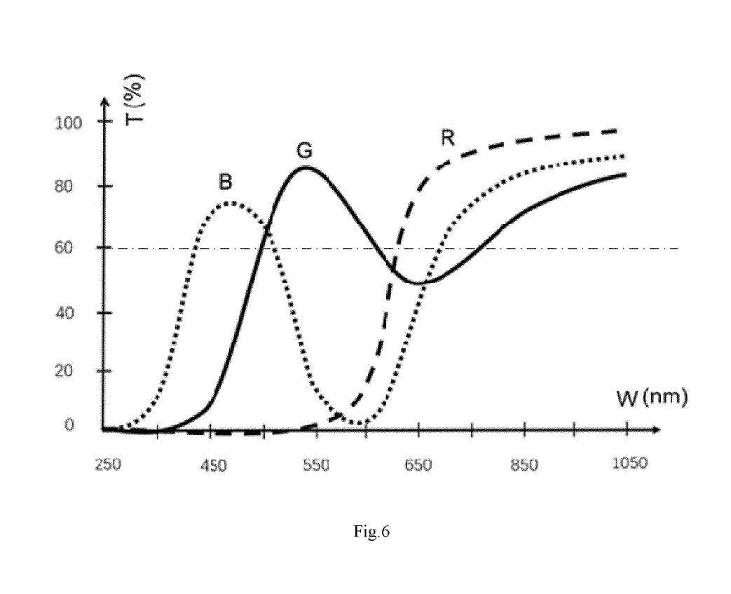

FIG. 6 shows a transmittance diagram of red filter, green filter and blue filter for light radiations with various wavelengths. As shown in FIG. 6, the transmittance for the three color filters is greater than 60%, in the infrared range, from 760 nm to 1000 nm.

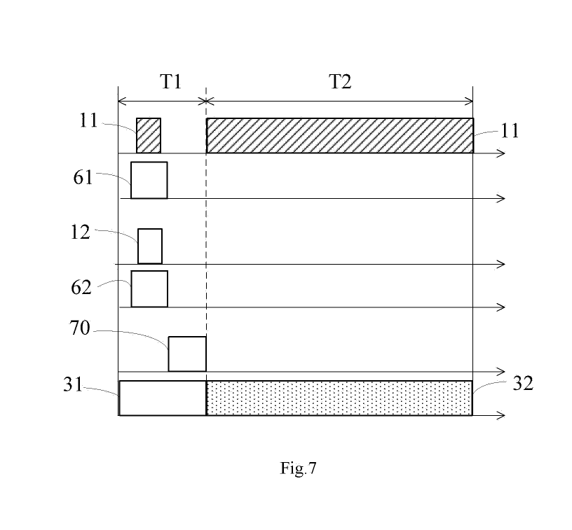

FIG. 7 shows a time sequence of a portable multispectral imaging and projecting device of one embodiment of the present disclosure. As shown in FIG. 7, a visible light projecting time pulse 11 represents a projecting time of the visible light sources, an infrared light projecting time pulse 12 represents a projecting time of the infrared source, a visible light image acquisition time pulse 61 represents the acquisition time of the visible light image, an infrared light image acquisition time pulse 62 represents the acquisition time of the infrared image, an image generating time pulse 71 represents the image generating time, an activating time pulse 31 represents the activating time of the liquid crystal valve without performing modulation processes and an activating time pulse 32 represents the activating time of the liquid crystal valve with performing modulation processes.

As shown in FIG. 7, during a time interval T1, a light source module including a plurality of visible light sources and at least one infrared light source is turned on to project visible light and infrared light, passing through a liquid crystal light valve, to a target area. In this embodiment, the liquid crystal light valve allows the visible light and the infrared light to pass through without modulation processes.

Moreover, when the light source module is turned on, a visible light image of the target area and an infrared light image of the target area are acquired, by a photoelectric sensor of an image acquiring module. When the light source module is turned off, an image is generated by a processor in response to the visible light image and the infrared light image.

During a time interval T2, as shown in FIG. 7, the visible light sources project the visible light to the target area again. The intensity of the visible light is then modulated, by the liquid crystal valve, in response to the image of the target area. The image presented by the modulated visible light is then projected to the target area. In this embodiment, the light intensity of the image is stronger than the intensity of the modulated visible light. In this embodiment, the image includes blood vessels pattern. In this embodiment, the infrared light includes a wavelength in a range of 760 nm to 1000 nm.

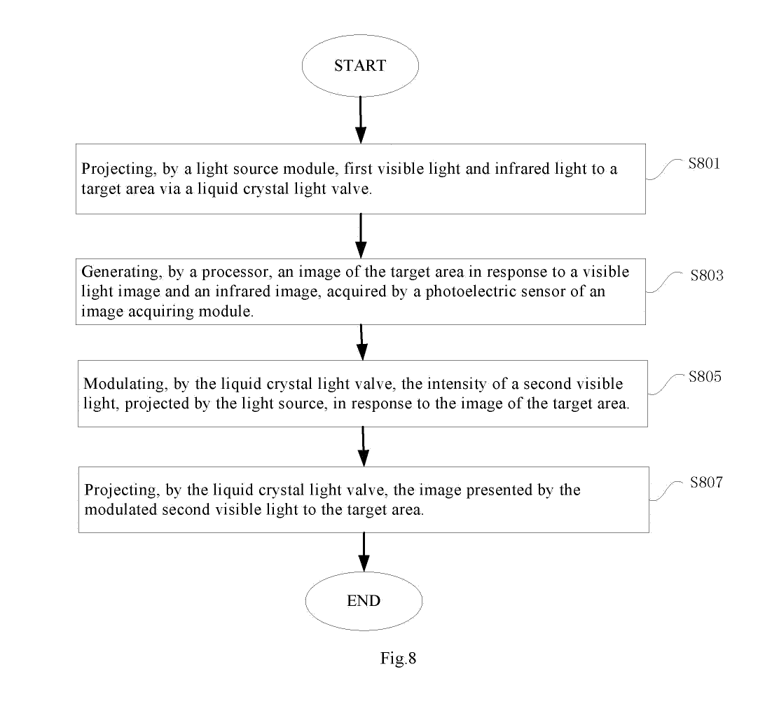

FIG. 8 shows a multispectral imaging and projecting method implemented on portable multispectral imaging and projecting device of FIG. 1. As shown in FIG. 8, in step S801, visible light and infrared are projected to a target area via a liquid crystal light valve and a lens module, wherein the visible light and the infrared are projected by a light source module. In step S803, an image is generated, by a processor, in response to a visible light image and an infrared image of the target area. In this embodiment, visible light image and an infrared image of the target area are acquired by an image acquiring module. In step S805, the intensity of a visible light, projected by the light source again, is modulated, by the liquid crystal light valve, in response to the image of the target area. In step S807, the image, e.g. the vessels image, presented by the modulated visible light is then projected, by the liquid crystal light valve, to the target area. In this embodiment, the light intensity of the image is stronger than the intensity of the modulated visible light.

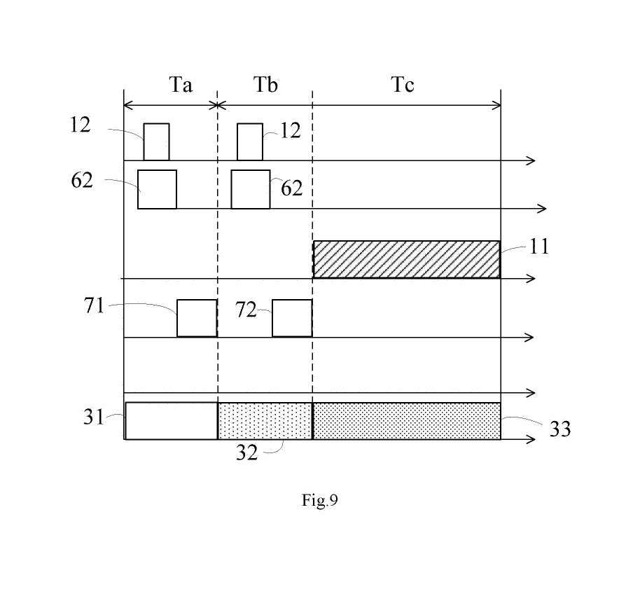

FIG. 9 shows a time sequence of a portable multispectral imaging and projecting device of one embodiment of the present disclosure. As shown in FIG. 9, during a time interval Ta, an infrared light projecting time pulse 90 represents a first projecting time of the infrared light source, an infrared image acquisition time pulse 91 represents a first acquisition time of a first infrared image, a first image processing time pulse 93 represents the first infrared image processing time, and an activating time pulse 95 represents a first activating time of the liquid crystal valve.

Therefore, during the time interval Ta, a light source module, including a plurality of visible light sources and at least one infrared light source, is turned on to project first infrared light, passing through a liquid crystal light valve, to a target area. The liquid crystal light valve allows the first infrared light to pass through without performing modulation process.

In this embodiment, when the light source module is turned on, a first infrared light image of the target area is acquired, by a photoelectric sensor of an image acquiring module. When the light source module is turned off, the first infrared image is processed, by a processor, and the processed first infrared image is then transmitted to the liquid crystal light valve.

In this embodiment, vessels pattern of the first infrared image is similar to vessels pattern of the processed first infrared image, but the vessels pattern of the processed first infrared image is larger than the vessels pattern of the first infrared image, which reduces offset of image alignment, image edge scattering and attenuation caused in infrared light projection during a time interval Tb. For example, in this embodiment, the edge of the vessels pattern of the processed first infrared image is distant from the edge of the vessels pattern of the first infrared image approximately 0.1 mm.

Moreover, during the time interval Tb, an infrared light projecting time pulse 92 represents the second projecting time of the infrared light source, an infrared light image acquisition time pulse 94 represents a second acquisition time of an infrared image, a processed second infrared image processing time pulse 96 represents a second infrared image processing time, and an activating time pulse 98 represents the second activating time of the liquid crystal valve.

Therefore, during the time interval Tb, the infrared light source is turned on to project the infrared light, passing through the liquid crystal light valve, to the target area again. In this embodiment, the intensity of the infrared light, passing through the liquid crystal light valve, is modulated in response to the processed first infrared image.

Moreover, when the infrared light source is turned on, second infrared image of the target area is acquired, by a photoelectric sensor of an image acquiring module. When the infrared light source is turned off, the second infrared image is processed by the processor and the processed second infrared image is then transmitted to the liquid crystal light valve. In this embodiment, the intensity of the processed second infrared image is greater than the intensity of the modulated infrared light.

During a time interval Tc, a visible light projecting time pulse 97 represents the projecting time of the visible light source, and an activating time pulse 99 represents the third activating time of the liquid crystal valve with second modulation process.

Therefore, during the time interval Tc, the visible light sources are turned on to project visible light, passing through the liquid crystal valve, to the target area. In this embodiment, the intensity of the visible light is modulated in response to the processed second infrared image. The processed second infrared image is then projected with the modulated visible light to the target area. In this embodiment, the processed second infrared image is presented by visible light. Moreover, the light intensity of the processed second infrared image is greater than the intensity of the modulated visible light. In this embodiment, the processed second infrared image includes a vessel pattern. In this embodiment, the infrared light includes a wavelength in a range of 760 nm to 1000 nm. In this embodiment, the processed second infrared image, presented by the modulated visible light, is projected to the target area by the liquid crystal light valve.

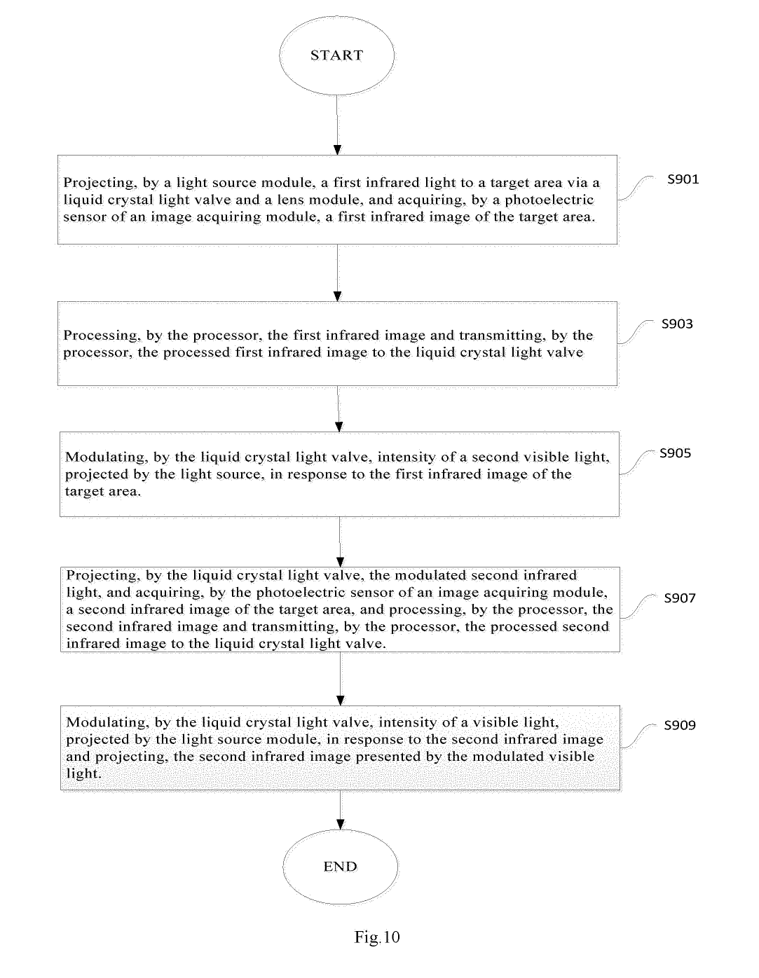

FIG. 10 shows a portable multispectral imaging and projecting method implemented on portable multispectral imaging and projecting device of one embodiment of the present disclosure. As shown in FIG. 10, in step S901, first infrared light is projected, by a light source module, via a liquid crystal light valve and a lens module to a target area, and a first infrared image of the target area is acquired by a photoelectric sensor of an image acquiring module. In this embodiment, the light source module includes a plurality of visible light sources and at least one infrared light source.

In step S903, the first infrared image of the target area is processed by the processor and the processed first infrared image is transmitted, by the image processor, to the liquid crystal light valve. Moreover, intensity of second infrared light, projected by the light source module, is modulated, by the liquid crystal light valve, in response to the processed first infrared image.

Moreover, in this embodiment, in order to ensuring completely cover the blood vessel pattern, and reducing misalignment, and minimize lateral light scattering caused by surrounding subcutaneous structures, when the processed first infrared image is stacked with the blood vessel pattern in the first infrared image, a blood vessel pattern in the processed first infrared image is central aligned to, and cover, the blood vessel pattern in the first infrared image and the dimension of the blood vessel pattern in the processed first infrared image is 0.1 mm to 0.5 mm larger than the vessel pattern in the first infrared image.

In step S905, the intensity of second infrared light, projected by the light source module, is modulated by the liquid crystal valve in response to the processed first infrared image.

In step S907, the processed first infrared image presented by the modulated second infrared light is projected, via the liquid crystal light valve and lens module, to the target area; a second infrared image of the target area is acquired by the photoelectric sensor of the image acquisition module. The second infrared image is processed by the processor and the processed second infrared image is transmitted to the liquid crystal light valve.

Moreover, in step S909, intensity of a visible light, projected by the light source module, is modulated, by the liquid crystal light valve, in response to the processed second infrared image, and the processed second infrared image presented by the modulated visible light is projected, via the liquid crystal valve and the lens module, to the target area.

Therefore, in this embodiment, after performing two consecutive infrared image acquisitions, degradation of image contrast caused by light scattering are reduced and the blood vessels pattern projected on the target area is enhanced significantly, which allow the users to accurately apply medical diagnoses or treatments.

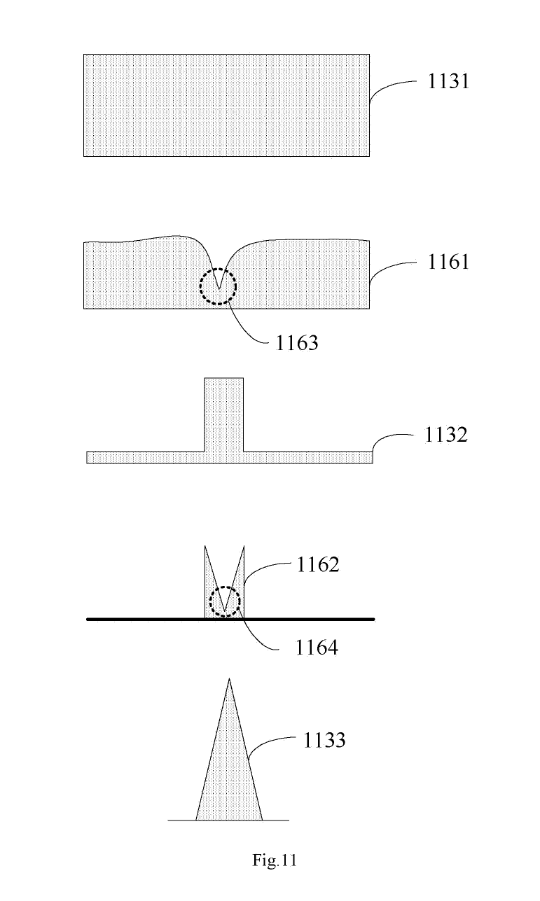

FIG. 11 shows infrared light intensity distribution diagrams of one embodiment of the present disclosure. As shown in FIG. 11, an intensity distribution diagram 1131 represents the infrared light intensity of the first infrared light radiation without modulation, e.g. spatial modulation, by the liquid crystal valve. An intensity distribution diagram 1161 represents the infrared light intensity of the first infrared image. As shown in FIG. 11, an absorption valley 1163 of the intensity distribution diagram 1161 indicates the location of the blood vessel.

Moreover, an intensity distribution diagram 1132 represents the infrared light intensity of the modulated second infrared light. As shown in FIG. 11, the infrared light intensity has peak intensity at the location of the blood vessel. An intensity distribution diagram 1162 represents the infrared light intensity of the second infrared image. As shown in FIG. 11, an absorption valley 1164 of the intensity distribution diagram 1162 is corresponding to the location of blood vessels. Comparing to intensity distribution of 1161, absorption valley 1164 appears to be sharper than absorption valley 1163, suggesting fewer laterals light scattering and therefore higher image contrast of blood vessel.

Finally, an intensity distribution diagram 1133 representing in high contrast the blood vessel image can be extracted from distribution diagram 1162.

What is claimed is:

1. A portable multispectral imaging and projecting device, comprising:
a liquid crystal light valve configured to modulate, in response to an image generated by a processor, intensity of light, emitted by a light source module and passing through the liquid crystal light valve, and project the modulated light and the image to a target area;
wherein the light source module includes a plurality of visible light sources and at least one infrared light source and the light source module is configured to project visible light and infrared light; and
wherein the image is generated in response to an infrared image and a visible light image, or the infrared image, wherein the images are acquired by a photoelectric sensor of an image acquiring module.

2. The portable multispectral imaging and projecting device of claim 1 further comprising a time controller configured to control the light source module to simultaneously or alternatively emit the light.

3. The portable multispectral imaging and projecting device of claim 1 further comprising a first lens module configured to focus the modulated light to the target area, wherein the center of the first lens module, the center of the light source module and the center of the liquid crystal light valve are aligned on an optical axis.

4. The portable multispectral imaging and projecting device of claim 1, wherein the photoelectric sensor is configured to acquire the infrared image of infrared light including a wavelength in a range of 760 nm to 1000 nm.

5. The portable multispectral imaging and projecting device of claim 1, wherein the liquid crystal light valve further includes a liquid crystal layer configured to modulate the intensity of light.

6. The portable multispectral imaging and projecting device of claim 5 further including a pixel array driven by thin film transistor (TFT) array, wherein the pixel array includes a plurality of pixels and each of the pixels is covered with a color filter which allows infrared light and a portion of a visible light passing through.

7. The portable multispectral imaging and projecting device of claim 1, wherein the photoelectric sensor includes a first imaging unit configured to acquire the visible light image and a second imaging unit configured to acquire the infrared image.

8. The portable multispectral imaging and projecting device of claim 7 further including a second lens module configured to focus visible light on the first imaging unit and focus infrared light on the second imaging unit.

9. The portable multispectral imaging and projecting device of claim 1, wherein the photoelectric sensor includes a charge-coupled device (CCD) imaging sensor, or a complementary metal oxide semiconductor (CMOS) imaging sensor, wherein the CMOS imaging sensor including a photodiode and at least one transistor.

10. The portable multispectral imaging and projecting device of claim 1, wherein the image acquiring module includes a CCD imaging sensor integrated with a semiconductor photoelectric conversion film, or a CMOS imaging sensor integrated with the semiconductor photoelectric conversion film.

11. The portable multispectral imaging and projecting device of claim 10, wherein the semiconductor photoelectric conversion film includes amorphous silicon or a metal oxide semiconductor.

12. A method of multispectral imaging and projecting implemented on a portable multispectral imaging and projecting device, the method comprising steps of:
projecting, by a light source module, first visible light and infrared light to a target area via a liquid crystal light valve;
generating, by a processor, an image of the target area in response to a visible light image and an infrared image, acquired by a photoelectric sensor of an image acquiring module;
modulating, by the liquid crystal light valve, the intensity of a second visible light, projected by the light source, in response to the image of the target area; and
projecting, by the liquid crystal light valve, the image presented by the modulated second visible light to the target area;
wherein the light source module includes a plurality of visible light sources and at least one infrared light source.

13. A method of multispectral imaging and projecting implemented on a portable multispectral imaging and projecting device, the method comprising steps of:
projecting, by a light source module, first infrared light to a target area via a liquid crystal light valve and a lens module, and acquiring, by a photoelectric sensor of an image acquiring module, a first infrared image of the target area, wherein the light source module includes a plurality of visible light sources and at least one infrared light source;
processing, by the processor, the first infrared image and transmitting, by the processor, the processed first infrared image to the liquid crystal light valve;
modulating, by the liquid crystal light valve, intensity of a second infrared light, projected by the light source module, in response to the processed first infrared image;
projecting, by the liquid crystal light valve and the lens module, the modulated second infrared light to the target area, and acquiring, by the photoelectric sensor of an image acquiring module, a second infrared image of the target area;
processing, by the processor, the second infrared image and transmitting, by the processor, the processed second infrared image to the liquid crystal light valve;
modulating, by the liquid crystal light valve, intensity of a visible light, projected by the light source module, in response to the processed second infrared image; and
projecting, by the liquid crystal light valve and the lens module, the processed second infrared image presented by the modulated visible light to the target area.

14. The method of claim 13, wherein image pattern in the processed first infrared image is central aligned to and larger enough to cover, the image pattern in the first infrared image.

15. The method of claim 14, wherein the dimension of the image pattern in the processed first infrared image is 0.1 mm to 0.5 mm larger than the image pattern in the first infrared image.
